SEQUENCE LISTING (1 sequence)

SEQ ID NO: 1 - protein, 10 aa, synthetic construct
  SITE 8: note = diphenylalanine
  SITE 10: note = amine group
KKWWKKWXKX

(19) United States
(12) Patent Application Publication
(10) Pub. No.: US 2025/0241976 A1
(43) Pub. Date: Jul. 31, 2025

(54) ADMINISTRATION OF AN ANTICANCER PEPTIDE

(71) Applicant: Verrica Pharmaceuticals, Inc., West Chester, PA (US)

(72) Inventors: Gary Goldenberg, West Chester, PA (US); Jayson Michael Rieger, Charlottesville, VA (US)

(73) Assignee: Verrica Pharmaceuticals, Inc., West Chester, PA (US)

(21) Appl. No.: 19/037,004

(22) Filed: Jan. 24, 2025

Related U.S. Application Data
(60) Provisional application No. 63/624,965, filed on Jan. 25, 2024.

(51) Int. Cl. A61K 38/08; A61K 9/00

(52) U.S. Cl. CPC A61K 38/08; A61K 9/0019

(57) ABSTRACT

The present disclosure is based on the development of improved clinical protocols for administering an anti-cancer peptide having the sequence of SEQ ID NO: 1, or a pharmaceutically acceptable salt thereof (“Peptide A”), to a subject (e.g., for the treatment of a skin cancer, such as basal cell carcinoma (BCC)). Methods provided herein allow for the safe and effective treatment of skin cancers, including BCC, in a minimally invasive manner. In certain embodiments, methods provided herein allow for administration of Peptide A to a skin cancer lesion (e.g., BCC lesion) on a subject with no significant treatment-related adverse events (TRAEs), e.g., TRAEs such as burning and/or pain at the injection site.

### RELATED APPLICATIONS

This application claims priority under 35 U.S.C. § 119 (c) to U.S. Provisional Patent Application, U.S. Ser. No. 63/624,965, filed Jan. 25, 2024, the entire contents of which is incorporated herein by reference.

### REFERENCE TO AN ELECTRONIC SEQUENCE LISTING

The contents of the electronic sequence listing (V029670014US01-SEQ-PJH.xml; Size: 2,670 bytes; and Date of Creation: Jan. 17, 2025) is herein incorporated by reference in its entirety.

### BACKGROUND

Skin cancer is the most commonly diagnosed cancer in the United States (US), with non-melanoma skin cancer (NMSC) being the most common malignancy in the US. See, e.g., Kantor, J. “The SCAR Scale (Scar Cosmesis Assessment and Rating Scale): Development and validation of a new outcome measure for postoperative scar assessment.” British Journal of Dermatology, 2016, 175(6), 1394-13961; Karimkhani, C. et al. “It's time for ‘keratinocyte carcinoma to place the term ‘nonmelanoma skin cancer.’” J. Am. Acad. Dermatol., 2015, 72(1), 186-187; and Madan V. et al. “Non-melanoma skin cancer.” The Lancet, 2010, 375(9715), 673-685.

National incidence estimates based on the most recent year that new statistics were available (2012) estimated more than 5.4 million cases of NMSC were treated in more than 3.3 million people in the US, exceeding the number of all other cases of human malignancies combined. See, e.g., Rogers H. W. et al. “Incidence Estimate of Nonmelanoma Skin Cancer (Keratinocyte Carcinomas) in the US Population, 2012.” JAMA Dermatol., 2015, 151(10), 1081; American Academy of Dermatology Association (AAD), Skin Cancer, Incidence Rates, February 2021; and Skin Cancer Foundation, Skin Cancer Facts and Statistics, February 2021. Data from the Medical Expenditure Panel Survey estimated an annual increase in treatment for NMSC (2002 to 2011) in US adults from 3.1 to 4.3 million. Sec, e.g., Guy G. P. et al. “Prevalence and Cost of Skin Cancer Treatment in the U.S., 2002-2006 and 2007-2011.” American Journal of Preventative Medicine, 2015, 48(2), 183-164.

Current estimates show that the approximately 80% of NMSC cases occur in people ≥60 years of age, and that the number of cases in this population could increase by an estimated 50% by 2030, illustrating the significant worldwide healthcare burden of NMSC, particularly in light-skinned populations. Sec, e.g., Diffey B. and Langtry J. “Skin cancer incidence and the ageing population.” British Journal of Dermatology, 2005, 153(3), 679-680; Karimkhani, C. et al., “It's time for ‘keratinocyte carcinoma’ to replace the term ‘nonmelanoma skin cancer.’ J. Am. Acad. Dermatol. 2015, 72(1), 186-187. While skin cancers have a relatively low mortality rate, the average annual total cost for skin cancer increased from 2006 to 2011 by 126.2%, from $3.6 billion to $8.1 billion. Sec, e.g., Guy, G. P. et al., “Prevalence and Costs of Skin Cancer Treatment in the U.S., 2002-2006 and 2007-2011.” American Journal of Preventive Medicine, 2015, 48(2), 183-187.

In other studies, similar increases were seen in basal cell carcinoma (BCC) incidence rates specifically. Sec, e.g., Sveinbjørnsson B. et al. “LTX-315: a first-in-class oncolytic peptide that reprograms the tumor microenvironment.” Future Med. Chem., 2017, 9(12), 1339-1344; Wu S. et al. “Basal-cell carcinoma incidence and associated risk factors in U.S. women and men.” Am. J. Epidemiol., 2013, 178(6), 890-897; Goldenberg G. et al. “Incidence and prevalence of basal cell carcinoma (BCC) and locally advanced BCC (LABCC) in a large commercially insured population in the United States: A retrospective cohort study.” Journal of the American Academy of Dermatology, 2016, 75(5), 957-966.c2; and Adalsteinsson J. A. et al. “Basal cell carcinoma: an emerging epidemic in women in Iceland.” British Journal of Dermatology 2020, 183(5):847-856.

Surgery is the most common treatment for BCC and includes procedures such as excision, curettage and electrodesiccation, and Mohs micrographic surgery (MMS). Both excision and curettage leave scarring of the treatment area; MMS aims to reduce scarring, particularly in treatment areas near the eye or other areas of the face, but is a complex, protracted surgery requiring specialized training. Some current non-invasive options for treating BCC, including topical and intradermal therapies, are limited by concerns relating to toxicity and safety. There is a need for new, non-invasive methods for treating BCC and other skin cancers that are safe and effective.

### SUMMARY

Peptides with anti-cancer activity have been described in, e.g., in International PCT Application Publication Nos. WO 2010/060497 and WO 2017/134175, the entire contents of each of which is incorporated herein by reference. For example, a peptide having the amino acid sequence: KKWWKKWDipK-NH₂ (SEQ ID NO: 1) has proven effective against skin cancers including basal cell carcinoma (BCC).

The present disclosure is based on the development of improved clinical protocols for administering an anti-cancer peptide having the sequence of SEQ ID NO: 1, or a pharmaceutically acceptable salt thereof (“Peptide A”), to a subject (e.g., for the treatment of a skin cancer, such as basal cell carcinoma (BCC)). Methods described herein comprise intradermal injection of Peptide A into a skin cancer lesion (e.g., BCC lesion) and therefore represent minimally invasive options for safely and effectively treating skin cancers, including BCC.

In certain embodiments, methods provided herein allow for the administration of Peptide A to a skin cancer lesion (e.g., BCC lesion) on a subject with no significant treatment-related adverse events (TRAEs), e.g., TRAEs such as burning and/or pain at the injection site.

In one aspect, provided herein are methods comprising administering a pharmaceutical composition comprising Peptide A to a subject, wherein the total volume of the pharmaceutical composition is divided between two subsequent administration steps (“partitioned administration”). For example, provided herein are methods of treating basal cell carcinoma (BCC) in a subject in need thereof, the methods comprising the following sequential steps:
(a) injecting intradermally 20%-40%, inclusive, of the total volume of a pharmaceutical composition comprising Peptide A into a BCC skin lesion of the subject;
(b) waiting between 5 and 60 minutes, inclusive; and
(c) injecting intradermally the remaining 60%-80%, inclusive, of the total volume of the pharmaceutical composition into the BCC skin lesion. In some embodiments, about 30% of the total volume of the pharmaceutical composition is injected into the BCC skin lesion in step (a); and the remaining about 70% is injected into the BCC skin lesion in step (c). In some embodiments, step (b) comprises waiting between 5 and 30 minutes, inclusive (e.g., between 15 and 30 minutes, inclusive).

In another aspect, provided herein are methods comprising administering a pharmaceutical composition comprising Peptide A to a subject, wherein the pharmaceutical composition is injected first into the center of a skin cancer lesion, followed by two or more injections into the periphery of the skin cancer lesion (“radial administration”). For example, provided herein are methods of treating basal cell carcinoma (BCC) in a subject in need thereof, the methods comprising the following sequential steps:
(a) injecting intradermally a portion of the total volume of a pharmaceutical composition comprising Peptide A into the center of a BCC skin lesion of the subject; and
(b) injecting intradermally the remaining portion of the total volume of the pharmaceutical composition into 2 or more sites at the periphery of the BCC skin lesion. In some embodiments, step (b) comprises injecting the remaining portion of the total volume of the pharmaceutical composition into 3 or more sites (e.g., 4 or more sites) at the periphery of the BCC skin lesion. In some embodiments, step (b) comprises injecting the remaining portion of the total volume of the pharmaceutical composition into 2-6 sites (e.g., 3-5 sites, such as 2, 3, 4, 5 or 6 sites) at the periphery of the BCC skin lesion. In some embodiments, the sites at the periphery of the BCC skin lesion are approximately equidistant from one another.

In yet another aspect, provided herein are methods that combine the partitioned administration and radial administration methods described herein. For example, provided herein are methods of treating basal cell carcinoma (BCC) in a subject in need thereof, the methods comprising the following sequential steps:
(a) injecting intradermally 20%-40%, inclusive, of the total volume of a pharmaceutical composition comprising Peptide A into the center of a BCC skin lesion of the subject;
(b) waiting between 5 and 60 minutes, inclusive; and
(c) injecting intradermally the remaining 60%-80%, inclusive, of the total volume of the pharmaceutical composition to 2 or more sites at the periphery of the BCC skin lesion. In some embodiments, about 30% of the total volume of the pharmaceutical composition is injected into the BCC skin lesion in step (a); and the remaining about 70% is injected into the BCC skin lesion in step (c). In some embodiments, step (b) comprises waiting between 5 and 30 minutes, inclusive (e.g., between 15 and 30 minutes, inclusive). In some embodiments, step (c) comprises injecting the remaining portion of the total volume of the pharmaceutical composition into 3 or more sites (e.g., 4 or more sites) at the periphery of the BCC skin lesion. In some embodiments, step (c) comprises injecting the remaining portion of the total volume of the pharmaceutical composition into 2-6 sites (e.g., 3-5 sites, such as 2, 3, 4, 5, or 6 sites) at the periphery of the BCC skin lesion. In some embodiments, the sites at the periphery of the BCC skin lesion are approximately equidistant from one another.

In some embodiments of any of the preceding methods, Peptide A is the acetate salt of KKWWKKWDipK-NH₂ (SEQ ID NO: 1).

In some embodiments of any of the preceding methods, the total volume of the pharmaceutical composition is from 100 μL to 1000 μL, inclusive (e.g., from 400 μL to 600 μL, inclusive, e.g., approximately 500 μL). In some embodiments, the total volume of the pharmaceutical composition comprises from 2 mg to 8 mg, inclusive, of Peptide A (e.g., approximately 8 mg of Peptide A). In some embodiments, the concentration of Peptide A in the pharmaceutical composition is from 10 mg/mL to 20 mg/mL, inclusive (e.g., from 14 mg/mL to 18 mg/mL, inclusive, e.g., approximately 16 mg/mL). In some embodiments, the pharmaceutical composition comprises Peptide A and normal saline.

In some embodiments, any of the preceding methods is performed once daily. In some embodiments, the method is performed once daily for three consecutive days. In some embodiments, the method is performed once daily for two consecutive days.

In some embodiments, the method is performed once daily for 2-3 consecutive days per week. In some embodiments, the method is performed once daily for two consecutive days, followed by five consecutive days of no treatment with Peptide A (e.g., once daily for two consecutive days per week). In some embodiments, the method is performed once daily for three consecutive days, followed by four consecutive days of no treatment with Peptide A (e.g., once daily for three consecutive days per week). In some embodiments, no more than 8 mg of Peptide A is administered to the subject per day.

In some embodiments of any of the preceding methods, the pharmaceutical composition is injected using a syringe with a 30-gauge needle (e.g., 30-gauge, 0.5 inch needle). In some embodiments, the pharmaceutical composition is injected using a syringe with a beveled needle. In some embodiments of any of the preceding methods, the skin cancer lesion (e.g., BCC skin lesion) is a flat lesion, and the pharmaceutical composition is injected at a 10- to 20-degree angle, inclusive. In some embodiments, the skin cancer lesion (e.g., BCC skin lesion) is a raised lesion, and the pharmaceutical composition is injected at a greater than 20-degree angle.

In some embodiments of any of the preceding methods, the skin cancer is non-advanced skin cancer (e.g., the BCC is non-advanced basal cell carcinoma). In some embodiments, the skin cancer lesion (e.g., BCC skin lesion) is from 0.5 cm to 2.0 cm, inclusive, in its longest diameter. In some embodiments, the skin cancer lesion is greater than 0.5 cm in its longest diameter.

In some embodiments of any of the preceding methods, the subject is a human. In some embodiments, the subject is a human 18 years of age or older.

Also provided herein are pharmaceutical compositions comprising Peptide A for use in any of the methods described herein. Additionally, provided herein are uses of pharmaceutical compositions comprising Peptide A as medicaments for the treatment of skin cancer (e.g., BCC) according to any of the methods described herein.

The details of some embodiments of the disclosure are set forth in the Detailed Description, as described below. Other embodiments of the disclosure will be apparent from the Definitions, Examples, Drawings, Abstract, and Claims.

### BRIEF DESCRIPTION OF THE DRAWINGS

The accompanying drawings, which are incorporated in and constitute a part of this specification, illustrate several embodiments of the disclosure and together with the description, provide non-limiting examples of the disclosure.

FIG. 1. Exemplary injection pattern for Peptide A using “clockface administration.” C=injection site at the approximate center of the skin cancer lesion; 12, 3, 6, 9=injection sites at the periphery of the skin cancer lesion that are approximately equidistant apart.

FIG. 2. Structure of Peptide A (SEQ ID NO: 1).

### DETAILED DESCRIPTION

The present disclosure is based on the development of improved clinical protocols for administering an anti-cancer peptide having the sequence of SEQ ID NO: 1, or a pharmaceutically acceptable salt thereof (“Peptide A”), to a subject (e.g., for the treatment of a skin cancer, such as basal cell carcinoma (BCC)). Methods described herein comprise intradermal injection of Peptide A into a skin cancer lesion (e.g., BCC lesion) and therefore represent minimally invasive options for safely and effectively treating skin cancers, including BCC. In certain embodiments, methods provided herein allow for the administration of Peptide A to a skin cancer lesion (e.g., BCC lesion) on a subject with no significant treatment-related adverse events (TRAEs), e.g., TRAEs such as burning and/or pain at the injection site.

### Administration of Peptide A

i. Partitioned Administration

In one aspect, provided herein are methods comprising administering a pharmaceutical composition comprising Peptide A to a subject, wherein the total volume of the pharmaceutical composition is administered to the subject over two subsequent administration steps (“partitioned administration”). For example, methods provided herein comprise administering a portion of the pharmaceutical composition (e.g., 20%-40%, inclusive, of the total volume), and then waiting a period of time (e.g., between 5 and 60 minutes, inclusive) before administering the remaining portion of the pharmaceutical composition (e.g., the remaining 60%-80%, inclusive, of the total volume).

Provided herein are methods of treating skin cancer (e.g., basal cell carcinoma (BCC)) in a subject in need thereof, the methods comprising the following sequential steps:
(a) injecting intradermally 20%-40%, inclusive, of the total volume of a pharmaceutical composition comprising Peptide A into a skin cancer lesion of the subject;
(b) waiting between 5 and 60 minutes, inclusive; and
(c) injecting intradermally the remaining 60%-80%, inclusive, of the total volume of the pharmaceutical composition into the skin cancer lesion.

In some embodiments, the skin cancer is basal cell carcinoma (BCC). Therefore, provided herein are methods of treating basal cell carcinoma (BCC) in a subject in need thereof, the methods comprises the following sequential steps:
(a) injecting intradermally 20%-40%, inclusive, of the total volume of a pharmaceutical composition comprising Peptide A into a BCC skin lesion of the subject;
(b) waiting between 5 and 60 minutes, inclusive; and
(c) injecting intradermally the remaining 60%-80%, inclusive, of the total volume of the pharmaceutical composition into the BCC skin lesion.

As described, in certain embodiments the methods comprise a step (a) of injecting intradermally 20%-40%, inclusive, of the total volume of a pharmaceutical composition comprising Peptide A into a skin cancer lesion (e.g., BCC skin lesion) of the subject. In some embodiments, 25%-35%, inclusive, of the total volume of the pharmaceutical composition is injected into the skin cancer lesion (e.g., BCC skin lesion) in step (a). In some embodiments, 28%-32%, inclusive, of the total volume of the pharmaceutical composition is injected into the skin cancer lesion (e.g., BCC skin lesion) in step (a). In some embodiments, about 30% of the total volume of the pharmaceutical composition is injected into the skin cancer lesion (e.g., BCC skin lesion) in step (a). In some embodiments, approximately 20%, 21%, 22%, 23%, 24%, 25%, 26%, 27%, 28%, 29%, 30%, 31%, 32%, 33%, 34%, 35%, 36%, 37%, 38%, 39%, or 40%, of the total volume of the pharmaceutical composition is injected into the skin cancer lesion (e.g., BCC skin lesion) in step (a).

As described, in certain embodiments the methods comprise a step (b) of waiting between 5 and 60 minutes, inclusive (i.e., after step (a) and before step (c), or between the two injections). In some embodiments, step (b) comprises waiting between 5 and 10 minutes, inclusive. In some embodiments, step (b) comprises waiting between 5 and 20 minutes, inclusive. In some embodiments, step (b) comprises waiting between 5 and 30 minutes, inclusive. In some embodiments, step (b) comprises waiting between 5 and 40 minutes, inclusive. In some embodiments, step (b) comprises waiting between 5 and 50 minutes, inclusive. In some embodiments, step (b) comprises waiting between 5 and 60 minutes, inclusive. In some embodiments, step (b) comprises waiting between 15 and 60 minutes, inclusive. In some embodiments, step (b) comprises waiting between 15 and 50 minutes, inclusive. In some embodiments, step (b) comprises waiting between 15 and 40 minutes, inclusive. In some embodiments, step (b) comprises waiting between 15 and 30 minutes, inclusive. In some embodiments, step (b) comprises waiting between 15 and 20 minutes, inclusive.

As described, in certain embodiments the methods comprise a step (c) of intradermally injecting the remaining 60%-80%, inclusive, of the total volume of the pharmaceutical composition into the skin cancer lesion (e.g., BCC skin lesion). In some embodiments, the remaining 65%-75%, inclusive, of the total volume of the pharmaceutical composition is injected into the skin cancer lesion (e.g., BCC skin lesion) in step (c). In some embodiments, the remaining 68%-72%, inclusive, of the total volume of the pharmaceutical composition is injected into the skin cancer lesion (e.g., BCC skin lesion) in step (c). In some embodiments, the remaining about 70% of the total volume of the pharmaceutical composition is injected into the skin cancer lesion (e.g., BCC skin lesion) in step (c). In some embodiments, approximately 60%, 61%, 62%, 63%, 64%, 65%, 66%, 67%, 68%, 69%, 70%, 71%, 72%, 73%, 74%, 75%, 76%, 77%, 78%, 79%, or 80%, of the total volume of the pharmaceutical composition is injected into the skin cancer lesion (e.g., BCC skin lesion) in step (c).

In certain embodiments, about 30% of the total volume of the pharmaceutical composition is intradermally injected into the skin cancer lesion (e.g., BCC skin lesion) in step (a); and the remaining about 70% is intradermally injected into skin cancer lesion (e.g., BCC skin lesion) in step (c).

In certain embodiments, about 30% of the total volume of the pharmaceutical composition is intradermally injected into the skin cancer lesion (e.g., BCC skin lesion) in step (a); the waiting time is between 5 and 30 minutes, inclusive, in step (b); and the remaining about 70% is intradermally injected into skin cancer lesion (e.g., BCC skin lesion) in step (c).

In certain embodiments, about 30% of the total volume of the pharmaceutical composition is intradermally injected into the skin cancer lesion (e.g., BCC skin lesion) in step (a); the waiting time is between 15 and 30 minutes, inclusive, in step (b); and the remaining about 70% is intradermally injected into skin cancer lesion (e.g., BCC skin lesion) in step (c).

In some embodiments, the total volume of the pharmaceutical composition is from 10 μL to 2000 μL, inclusive (e.g., comprising from 2 mg to 8 mg, inclusive, of Peptide A; from 4 mg to 8 mg, inclusive, of Peptide A; from 6 mg to 8 mg, inclusive, of Peptide A; or approximately 8 mg of Peptide A). In some embodiments, the total volume of the pharmaceutical composition is from 10 μL to 1000 μL, inclusive (e.g., comprising from 2 mg to 8 mg, inclusive, of Peptide A; from 4 mg to 8 mg, inclusive, of Peptide A; from 6 mg to 8 mg, inclusive, of Peptide A; or approximately 8 mg of Peptide A). In some embodiments, the total volume of the pharmaceutical composition is from 100 μL to 1000 μL, inclusive (e.g., comprising from 2 mg to 8 mg, inclusive, of Peptide A; from 4 mg to 8 mg, inclusive, of Peptide A; from 6 mg to 8 mg, inclusive, of Peptide A; or approximately 8 mg of Peptide A). In some embodiments, the total volume of the pharmaceutical composition is from 200 μL to 800 μL, inclusive (e.g., comprising from 2 mg to 8 mg, inclusive, of Peptide A; from 4 mg to 8 mg, inclusive, of Peptide A; from 6 mg to 8 mg, inclusive, of Peptide A; or approximately 8 mg of Peptide A). In some embodiments, the total volume of the pharmaceutical composition is from 400 μL to 600 μL, inclusive (e.g., comprising from 2 mg to 8 mg, inclusive, of Peptide A; from 4 mg to 8 mg, inclusive, of Peptide A; from 6 mg to 8 mg, inclusive, of Peptide A; or approximately 8 mg of Peptide A). In some embodiments, the total volume of the pharmaceutical composition is approximately 500 μL (e.g., comprising from 2 mg to 8 mg, inclusive, of Peptide A; from 4 mg to 8 mg, inclusive, of Peptide A; from 6 mg to 8 mg, inclusive, of Peptide A; or approximately 8 mg of Peptide A). In some embodiments, the total volume of the pharmaceutical composition is approximately 500 μL and comprises approximately 8 mg of Peptide A.

In certain embodiments, the method of partitioned administration (including the sequential steps (a)-(c)) is performed once daily. In certain embodiments, the method is performed once daily for 2-3 consecutive days per week.

In certain embodiments, the method is performed once daily for two consecutive days. In certain embodiments, the method is performed once daily for two consecutive days, followed by one or more consecutive days (e.g., 1, 2, 3, 4, 5, 6, 7 consecutive days) of no treatment with Peptide A (referred to as a “treatment cycle”). In certain embodiments, the method is performed once daily for two consecutive days, followed by five consecutive days of no treatment with Peptide A (e.g., once daily for two consecutive days per week). In certain embodiments, the method is performed once daily for three consecutive days. In certain embodiments, the method is performed once daily for three consecutive days, followed by one or more consecutive days (e.g., 1, 2, 3, 4, 5, 6, or 7 consecutive days) of no treatment with Peptide A (referred to as a “treatment cycle”). In certain embodiments, the method is performed once daily for three consecutive days, followed by four consecutive days of no treatment with Peptide A (e.g., once daily for three consecutive days per week). In certain embodiments, no more than 8 mg/day of Peptide A is administered to the subject. In certain embodiments, a treatment cycle is be repeated one or more times (e.g., 1, 2, 3, 4, 5, 6, or 7 more times) until a desired outcome is achieved, e.g., until the skin cancer lesion has necrosed, reduced in size, or cleared.

Also provided herein are pharmaceutical compositions comprising Peptide A for use in any of the methods of partitioned administration described herein. Additionally, provided herein are uses of pharmaceutical compositions comprising Peptide A as medicaments for treating skin cancer (e.g., BCC) according to any of the methods of partitioned administration described herein.

ii. Radial Administration

In one aspect, provided herein are methods comprising administering a pharmaceutical composition comprising Peptide A to a subject, wherein the pharmaceutical composition is injected first into the center of a skin cancer lesion, followed by two or more injections into the periphery of the skin cancer lesion (“radial administration”).

Provided herein are methods of treating skin cancer (e.g., basal cell carcinoma (BCC)) in a subject in need thereof, the methods comprising the following sequential steps:
(a) injecting intradermally a portion of the total volume of a pharmaceutical composition comprising Peptide A into the center of a skin cancer lesion (e.g., BCC skin lesion) of the subject; and
(b) injecting intradermally the remaining portion of the total volume of the pharmaceutical composition into 2 or more sites at the periphery of the skin cancer lesion (e.g., BCC skin lesion).

As described, in certain embodiments, the skin cancer is basal cell carcinoma (BCC). Provided herein are methods of treating basal cell carcinoma (BCC) in a subject in need thereof, the methods comprising the following sequential steps:
(a) injecting intradermally a portion of the total volume of a pharmaceutical composition comprising Peptide A into the center of a BCC skin lesion of the subject; and
(b) injecting intradermally the remaining portion of the total volume of the pharmaceutical composition into 2 or more sites at the periphery of the BCC skin lesion.

As described, in certain embodiments the methods comprise a step (a) of injecting intradermally a portion of the total volume of the pharmaceutical composition into the center of the skin cancer lesion (e.g., BCC skin lesion). The “center” of the skin cancer lesion is a point in the approximate middle of the lesion as approximated by a physician using sound medical judgement. The exact center of a circle is the point in the middle of the circle which is equidistant from any point on the circumference of the circle. Because skin cancer lesions (e.g., BCC skin lesions) are rarely perfectly circular, a person of ordinary skill in the art would understand that the “center” of the skin cancer lesion is a point in the approximate middle of the lesion as approximated by a physician.

As described, in certain embodiments the methods comprise a step (b) of injecting intradermally the remaining portion of the total volume of the pharmaceutical composition into 2 or more sites at the periphery of the skin cancer lesion (e.g., BCC skin lesion). The “periphery” of the skin cancer lesion is the area between the approximate center of the lesion and the edges (i.e., the circumference) of the lesion.

In some embodiments, step (b) comprises injecting the remaining portion of the total volume of the pharmaceutical composition into 3 or more sites at the periphery of the skin cancer lesion (e.g., BCC skin lesion). In some embodiments, step (b) comprises injecting the remaining portion of the total volume of the pharmaceutical composition into 4 or more sites at the periphery of the skin cancer lesion (e.g., BCC skin lesion). In some embodiments, step (b) comprises injecting the remaining portion of the total volume of the pharmaceutical composition into 5 or more sites at the periphery of the skin cancer lesion (e.g., BCC skin lesion). In some embodiments, step (b) comprises injecting the remaining portion of the total volume of the pharmaceutical composition into 2-6 sites at the periphery of the skin cancer lesion (e.g., BCC skin lesion). In some embodiments, step (b) comprises injecting the remaining portion of the total volume of the pharmaceutical composition into 3-5 sites at the periphery of the skin cancer lesion (e.g., BCC skin lesion). In some embodiments, step (b) comprises injecting the remaining portion of the total volume of the pharmaceutical composition into 4 sites at the periphery of the skin cancer lesion (e.g., BCC skin lesion).

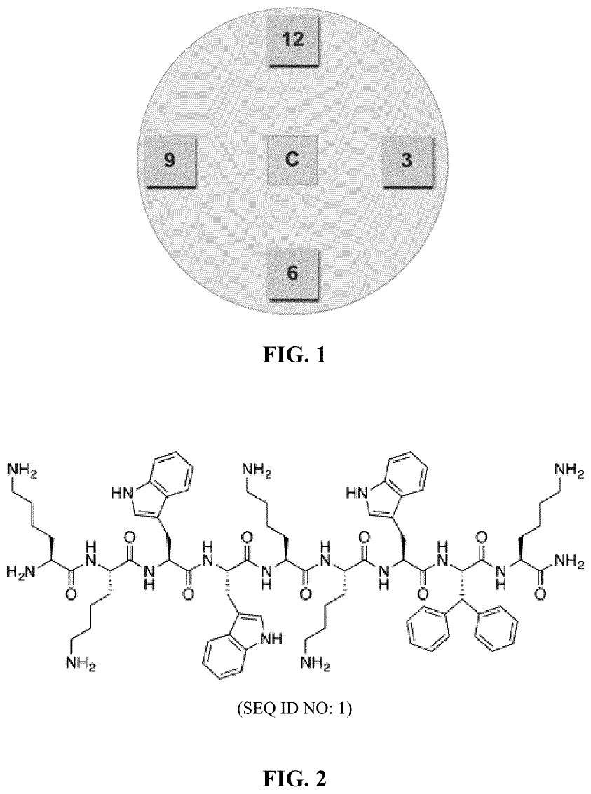

In some embodiments, the injection sites at the periphery of the skin cancer lesion (e.g., BCC skin lesion) in step (b) are approximately equidistant from one another. For example, in certain embodiments, step (b) comprises injecting the remaining portion of the total volume of the pharmaceutical composition into 4 sites at the periphery of the skin cancer lesion (e.g., BCC skin lesion), wherein the 4 sites are approximately equidistance from one another (i.e., corresponding to 12 o'clock, 3 o'clock, 6 o'clock, and 9 o'clock on a clock face, i.e., corresponding approximately to sites 12, 3, 6, and 9 indicated in FIG. 1). This administration pattern is referred to as “clockface administration.”

In some embodiments, step (b) comprises injecting the remaining portion of the total volume of the pharmaceutical composition into 2 or more sites (e.g., 2, 3, 4, 5, or 6 sites) at the periphery of the skin cancer lesion (e.g., BCC skin lesion) in a clockwise fashion (“clockwise administration”). In some embodiments, step (b) comprises injecting the remaining portion of the total volume of the pharmaceutical composition into 2 or more sites (e.g., 2, 3, 4, 5, or 6 sites) at the periphery of the skin cancer lesion (e.g., BCC skin lesion) in a counter-clockwise fashion (“counter-clockwise administration”). In other embodiments, no particular order is preferred.

In certain embodiments, step (a) comprises injecting a portion of the total volume of the pharmaceutical composition into the center of the BCC skin lesion; and step (b) comprises injecting the remaining portion of the total volume of the pharmaceutical composition into 4 or more sites at the periphery of the BCC skin lesion, wherein the 4 or more sites are approximately equidistant from one another.

In some embodiments, the remaining portion of the total volume is divided equally among the 2 or more sites (e.g., 2, 3, 4, 5, or 6 sites) at the periphery of the skin cancer lesion (e.g., BCC skin lesion) in step (b).

In some embodiments, the total volume of the pharmaceutical composition is from 10 μL to 2000 μL, inclusive (e.g., comprising from 2 mg to 8 mg, inclusive, of Peptide A; from 4 mg to 8 mg, inclusive, of Peptide A; from 6 mg to 8 mg, inclusive, of Peptide A; or approximately 8 mg of Peptide A). In some embodiments, the total volume of the pharmaceutical composition is from 10 μL to 1000 μL, inclusive (e.g., comprising from 2 mg to 8 mg, inclusive, of Peptide A; from 4 mg to 8 mg, inclusive, of Peptide A; from 6 mg to 8 mg, inclusive, of Peptide A; or approximately 8 mg of Peptide A). In some embodiments, the total volume of the pharmaceutical composition is from 100 μL to 1000 μL, inclusive (e.g., comprising from 2 mg to 8 mg, inclusive, of Peptide A; from 4 mg to 8 mg, inclusive, of Peptide A; from 6 mg to 8 mg, inclusive, of Peptide A; or approximately 8 mg of Peptide A). In some embodiments, the total volume of the pharmaceutical composition is from 200 μL to 800 μL, inclusive (e.g., comprising from 2 mg to 8 mg, inclusive, of Peptide A; from 4 mg to 8 mg, inclusive, of Peptide A; from 6 mg to 8 mg, inclusive, of Peptide A; or approximately 8 mg of Peptide A). In some embodiments, the total volume of the pharmaceutical composition is from 400 μL to 600 μL, inclusive (e.g., comprising from 2 mg to 8 mg, inclusive, of Peptide A; from 4 mg to 8 mg, inclusive, of Peptide A; from 6 mg to 8 mg, inclusive, of Peptide A; or approximately 8 mg of Peptide A). In some embodiments, the total volume of the pharmaceutical composition is approximately 500 μL (e.g., comprising from 2 mg to 8 mg, inclusive, of Peptide A; from 4 mg to 8 mg, inclusive, of Peptide A; from 6 mg to 8 mg, inclusive, of Peptide A; or approximately 8 mg of Peptide A). In some embodiments, the total volume of the pharmaceutical composition is approximately 500 μL and comprises approximately 8 mg of Peptide A.

In certain embodiments, the method of radial administration (including sequential steps (a) and (b)) is performed once daily. In certain embodiments, the method is performed once daily for 2-3 consecutive days per week.

In certain embodiments, the method is performed once daily for two consecutive days. In certain embodiments, the method is performed once daily for two consecutive days, followed by one or more consecutive days (e.g., 1, 2, 3, 4, 5, 6, or 7 consecutive days) of no treatment with Peptide A (referred to as a “treatment cycle”). In certain embodiments, the method is performed once daily for two consecutive days, followed by five consecutive days of no treatment with Peptide A (e.g., once daily for two consecutive days per week). In certain embodiments, the method is performed once daily for three consecutive days. In certain embodiments, the method is performed once daily for three consecutive days, followed by one or more consecutive days (e.g., 1, 2, 3, 4, 5, 6, or 7 consecutive days) of no treatment with Peptide A (referred to as a “treatment cycle”). In certain embodiments, the method is performed once daily for three consecutive days, followed by four consecutive days of no treatment with Peptide A (e.g., once daily for three consecutive days per week). In certain embodiments, no more than 8 mg/day of Peptide A is administered to the subject. In certain embodiments, a treatment cycle is be repeated one or more times (e.g., 1, 2, 3, 4, 5, 6, or 7 more times) until a desired outcome is achieved, e.g., until the skin cancer lesion has necrosed, reduced in size, or cleared.

Also provided herein are pharmaceutical compositions comprising Peptide A for use in any of the methods of radial administration described herein. Additionally, provided herein are uses of pharmaceutical compositions comprising Peptide A as medicaments for treating skin cancer (e.g., BCC) according to any of the methods of radial administration described herein.

iii. Partitioned and Radial Administration

In another aspect, provided herein are methods that combine the partitioned administration and radial administration methods described above. In certain embodiments, methods provided herein allow for administration of Peptide A to a skin cancer lesion (e.g., BCC lesion) on a subject with no significant treatment-related adverse events (TRAEs), e.g., TRAEs such as burning and/or pain at the injection site.

For instance, provided herein are methods of treating skin cancer (e.g., basal cell carcinoma (BCC)) in a subject in need thereof, the methods comprising the following sequential steps:
(a) injecting intradermally 20%-40%, inclusive, of the total volume of a pharmaceutical composition comprising Peptide A into the center of a skin cancer lesion (e.g., BCC skin lesion) of the subject;
(b) waiting between 5 and 60 minutes, inclusive; and
(c) injecting intradermally the remaining 60%-80%, inclusive, of the total volume of the pharmaceutical composition to 2 or more sites at the periphery of the skin cancer lesion (e.g., BCC skin lesion).

As described, in certain embodiments the skin cancer is basal cell carcinoma. Provided herein are methods of treating basal cell carcinoma (BCC) in a subject in need thereof, the methods comprising the following sequential steps:
(a) injecting intradermally 20%-40%, inclusive, of the total volume of a pharmaceutical composition comprising Peptide A into the center of a BCC skin lesion of the subject;
(b) waiting between 5 and 60 minutes, inclusive; and
(c) injecting intradermally the remaining 60%-80%, inclusive, of the total volume of the pharmaceutical composition to 2 or more sites at the periphery of the BCC skin lesion.

As described, in certain embodiments the methods comprise a step (a) of injecting intradermally 20%-40%, inclusive, of the total volume of the pharmaceutical composition into the center of the skin cancer lesion (e.g., BCC skin lesion). In some embodiments, 25%-35%, inclusive, of the total volume of the pharmaceutical composition is injected into the center of the skin cancer lesion (e.g., BCC skin lesion) in step (a). In some embodiments, 28%-32%, inclusive, of the total volume of the pharmaceutical composition is injected into the center of the skin cancer lesion (e.g., BCC skin lesion) in step (a). In some embodiments, about 30% of the total volume of the pharmaceutical composition is injected into the center of the skin cancer lesion (e.g., BCC skin lesion) in step (a). In some embodiments, about 20%, 21%, 22%, 23%, 24%, 25%, 26%, 27%, 28%, 29%, 30%, 31%, 32%, 33%, 34%, 35%, 36%, 37%, 38%, 39%, or 40%, of the total volume of the pharmaceutical composition is injected into the center of the skin cancer lesion (e.g., BCC skin lesion) in step (a).

As described, in certain embodiments the methods comprise a step (b) of waiting between 5 and 60 minutes, inclusive (i.e., after step (a) and before step (c)). In some embodiments, step (b) comprises waiting between 5 and 10 minutes, inclusive. In some embodiments, step (b) comprises waiting between 5 and 20 minutes, inclusive. In some embodiments, step (b) comprises waiting between 5 and 30 minutes, inclusive. In some embodiments, step (b) comprises waiting between 5 and 40 minutes, inclusive. In some embodiments, step (b) comprises waiting between 5 and 50 minutes, inclusive. In some embodiments, step (b) comprises waiting between 5 and 60 minutes, inclusive. In some embodiments, step (b) comprises waiting between 15 and 60 minutes, inclusive. In some embodiments, step (b) comprises waiting between 15 and 50 minutes, inclusive. In some embodiments, step (b) comprises waiting between 15 and 40 minutes, inclusive. In some embodiments, step (b) comprises waiting between 15 and 30 minutes, inclusive. In some embodiments, step (b) comprises waiting between 15 and 20 minutes, inclusive.

As described, in certain embodiments the methods comprise a step (c) of injecting intradermally the remaining 60%-80%, inclusive, of the total volume of the pharmaceutical composition into 2 or more sites (e.g., 2, 3, 4, 5, 6 sites) at the periphery of the skin cancer lesion (e.g., BCC skin lesion). In some embodiments, the remaining 65%-75%, inclusive, of the total volume of the pharmaceutical composition is injected into 2 or more sites (e.g., 2, 3, 4, 5, 6 sites) at the periphery of the skin cancer lesion (e.g., BCC skin lesion) in step (c). In some embodiments, the remaining 68%-72%, inclusive, of the total volume of the pharmaceutical composition is injected into 2 or more sites (e.g., 2, 3, 4, 5, 6 sites) at the periphery of the skin cancer lesion (e.g., BCC skin lesion) in step (c). In some embodiments, the remaining about 70% of the total volume of the pharmaceutical composition is injected into 2 or more sites (e.g., 2, 3, 4, 5, 6 sites) at the periphery of the skin cancer lesion (e.g., BCC skin lesion) in step (c). In some embodiments, approximately 60%, 61%, 62%, 63%, 64%, 65%, 66%, 67%, 68%, 69%, 70%, 71%, 72%, 73%, 74%, 75%, 76%, 77%, 78%, 79%, or 80%, of the total volume of the pharmaceutical composition is injected into 2 or more sites (e.g., 2, 3, 4, 5, 6 sites) at the periphery of the skin cancer lesion (e.g., BCC skin lesion) in step (c).

In some embodiments, step (c) comprises injecting the remaining portion of the total volume of the pharmaceutical composition into 3 or more sites at the periphery of the skin cancer lesion (e.g., BCC skin lesion). In some embodiments, step (c) comprises injecting the remaining portion of the total volume of the pharmaceutical composition into 4 or more sites at the periphery of the skin cancer lesion (e.g., BCC skin lesion). In some embodiments, step (c) comprises injecting the remaining portion of the total volume of the pharmaceutical composition into 5 or more sites at the periphery of the skin cancer lesion (e.g., BCC skin lesion). In some embodiments, step (c) comprises injecting the remaining portion of the total volume of the pharmaceutical composition into 2-6 sites at the periphery of the skin cancer lesion (e.g., BCC skin lesion). In some embodiments, step (c) comprises injecting the remaining portion of the total volume of the pharmaceutical composition into 3-5 sites at the periphery of the skin cancer lesion (e.g., BCC skin lesion). In some embodiments, step (c) comprises injecting the remaining portion of the total volume of the pharmaceutical composition into 4 sites at the periphery of the skin cancer lesion (e.g., BCC skin lesion).

In some embodiments, the remaining portion of the total volume is divided equally between the 2 or more sites (e.g., 2, 3, 4, 5, or 6 sites) at the periphery of the skin cancer lesion (e.g., BCC skin lesion) in step (c).

In some embodiments, the injection sites at the periphery of the skin cancer lesion (e.g., BCC skin lesion) in step (c) are approximately equidistant from one another. For example, in certain embodiments, step (c) comprises clockface administration into 4 sites at the periphery of the skin cancer lesion (e.g., BCC skin lesion). Sec, e.g., FIG. 1.

In some embodiments, step (c) comprises clockwise administration into the periphery of the skin cancer lesion (e.g., BCC skin lesion). In some embodiments, step (c) comprises counter-clockwise administration into the periphery of the skin cancer lesion (e.g., BCC skin lesion). In other embodiments, no particular order is preferred.

In certain embodiments, about 30% of the total volume of the pharmaceutical composition is injected intradermally into the center of the skin cancer lesion (e.g., BCC skin lesion) in step (a); and the remaining about 70% is injected intradermally into 2 or more sites (e.g., 2, 3, 4, 5, or 6 sites) at the periphery of skin cancer lesion (e.g., BCC skin lesion) in step (c). In some embodiments, the 2 or more sites (e.g., 2, 3, 4, 5, or 6 sites) at the periphery of skin cancer lesion (e.g., BCC skin lesion) are approximately equidistant from one another.

In certain embodiments, about 30% of the total volume of the pharmaceutical composition is injected intradermally into the center of the skin cancer lesion (e.g., BCC skin lesion) in step (a); the waiting time is between 5 and 30 minutes, inclusive, in step (b); and the remaining about 70% is injected intradermally into 2 or more sites (e.g., 2, 3, 4, 5, or 6 sites) at the periphery of skin cancer lesion (e.g., BCC skin lesion) in step (c). In some embodiments, the 2 or more sites (e.g., 2, 3, 4, 5, or 6 sites) at the periphery of skin cancer lesion (e.g., BCC skin lesion) are approximately equidistant from one another.

In certain embodiments, about 30% of the total volume of the pharmaceutical composition is injected intradermally into the center of the skin cancer lesion (e.g., BCC skin lesion) in step (a); the waiting time is between 15 and 30 minutes, inclusive, in step (b); and the remaining about 70% is injected intradermally into 2 or more sites (e.g., 2, 3, 4, 5, or 6 sites) at the periphery of skin cancer lesion (e.g., BCC skin lesion) in step (c). In some embodiments, the 2 or more sites (e.g., 2, 3, 4, 5, or 6 sites) at the periphery of skin cancer lesion (e.g., BCC skin lesion) are approximately equidistant from one another.

In some embodiments, the total volume of the pharmaceutical composition is from 10 μL to 2000 μL, inclusive (e.g., comprising from 2 mg to 8 mg, inclusive, of Peptide A; from 4 mg to 8 mg, inclusive, of Peptide A; from 6 mg to 8 mg, inclusive, of Peptide A; or approximately 8 mg of Peptide A). In some embodiments, the total volume of the pharmaceutical composition is from 10 μL to 1000 μL, inclusive (e.g., comprising from 2 mg to 8 mg, inclusive, of Peptide A; from 4 mg to 8 mg, inclusive, of Peptide A; from 6 mg to 8 mg, inclusive, of Peptide A; or approximately 8 mg of Peptide A). In some embodiments, the total volume of the pharmaceutical composition is from 100 μL to 1000 μL, inclusive (e.g., comprising from 2 mg to 8 mg, inclusive, of Peptide A; from 4 mg to 8 mg, inclusive, of Peptide A; from 6 mg to 8 mg, inclusive, of Peptide A; or approximately 8 mg of Peptide A). In some embodiments, the total volume of the pharmaceutical composition is from 200 μL to 800 μL, inclusive (e.g., comprising from 2 mg to 8 mg, inclusive, of Peptide A; from 4 mg to 8 mg, inclusive, of Peptide A; from 6 mg to 8 mg, inclusive, of Peptide A; or approximately 8 mg of Peptide A). In some embodiments, the total volume of the pharmaceutical composition is from 400 μL to 600 μL, inclusive (e.g., comprising from 2 mg to 8 mg, inclusive, of Peptide A; from 4 mg to 8 mg, inclusive, of Peptide A; from 6 mg to 8 mg, inclusive, of Peptide A; or approximately 8 mg of Peptide A). In some embodiments, the total volume of the pharmaceutical composition is approximately 500 μL (e.g., comprising from 2 mg to 8 mg, inclusive, of Peptide A; from 4 mg to 8 mg, inclusive, of Peptide A; from 6 mg to 8 mg, inclusive, of Peptide A; or approximately 8 mg of Peptide A). In some embodiments, the total volume of the pharmaceutical composition is approximately 500 μL and comprises approximately 8 mg of Peptide A.

In certain embodiments, the method combining partitioned administration and radial administration (including sequential steps (a)-(c)) is performed once daily. In certain embodiments, the method is performed once daily for 2-3 consecutive days per week.

In certain embodiments, the method is performed once daily for two consecutive days. In certain embodiments, the method is performed once daily for two consecutive days, followed by one or more consecutive days (e.g., 1, 2, 3, 4, 5, 6, 7 consecutive days) of no treatment with Peptide A (referred to as a “treatment cycle”). In certain embodiments, the method is performed once daily for two consecutive days, followed by five consecutive days of no treatment with Peptide A (e.g., once daily for two consecutive days per week). In certain embodiments, the method is performed once daily for three consecutive days. In certain embodiments, the method is performed once daily for three consecutive days, followed by one or more consecutive days (e.g., 1, 2, 3, 4, 5, 6, 7 consecutive days) of no treatment with Peptide A (referred to as a “treatment cycle”). In certain embodiments, the method is performed once daily for three consecutive days, followed by four consecutive days of no treatment with Peptide A (e.g., once daily for three consecutive days per week). In certain embodiments, no more than 8 mg/day of Peptide A is administered to the subject. In certain embodiments, a treatment cycle is be repeated one or more times (e.g., 1, 2, 3, 4, 5, 6, or 7 more times) until a desired outcome is achieved, e.g., until the skin cancer lesion has necrosed, reduced in size, or cleared.

Also provided herein are pharmaceutical compositions comprising Peptide A for use in any of the methods combining partitioned and radial administration described herein. Additionally, provided herein are uses of pharmaceutical compositions comprising Peptide A as medicaments for treating skin cancer (e.g., BCC) according to any of the methods combining partitioned and radial administration described herein.

### Peptide A

Methods described herein comprise administering to a subject a pharmaceutical composition comprising Peptide A. “Peptide A” as described herein is a peptide of the following amino acid sequence:
KKWWKKWDipK-NH₂ (SEQ ID NO: 1),
   or a pharmaceutically acceptable salt thereof.

Peptide A is described in, e.g., International PCT Application Publication Nos. WO 2010/060497 and WO 2017/134175, the entire contents of each of which is incorporated herein by reference. It is understood that in SEQ ID NO: 1, each K is the amino acid lysine, each W is the amino acid tryptophan, and Dip is the amino acid diphenylalanine. The structure of Peptide A is shown below:

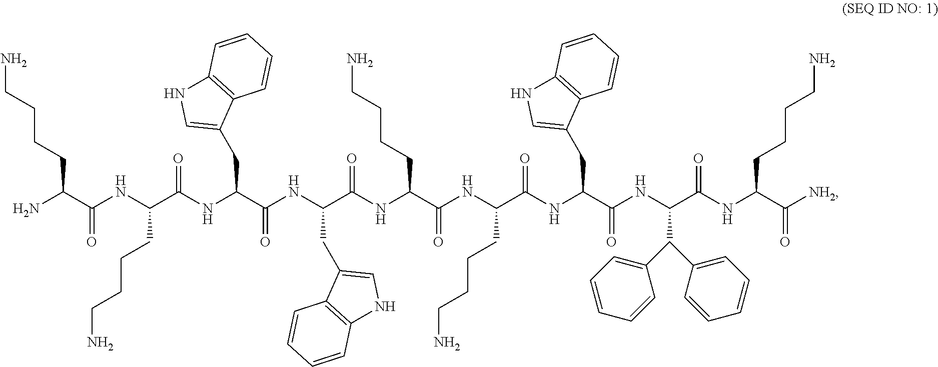 [SMILES: NCCCC[C@H](NC(=O)[C@@H](NC(=O)[C@H](Cc1c[nH]c2ccccc12)NC(=O)[C@H](CCCCN)NC(=O)[C@H](CCCCN)NC(=O)[C@H](Cc1c[nH]c2ccccc12)NC(=O)[C@H](Cc1c[nH]c2ccccc12)NC(=O)[C@H](CCCCN)NC(=O)[C@@H](N)CCCCN)C(c1ccccc1)c1ccccc1)C(N)=O]

or a pharmaceutically acceptable salt thereof.

As described, Peptide A may be a pharmaceutically acceptable salt of the peptide of SEQ ID NO: 1. In some embodiments, Peptide A is the acetate salt of the peptide of SEQ ID NO: 1.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of the present disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

Peptide A is a de novo designed oncolytic peptide with chemotherapeutic activity based the host-defense peptide lactoferricin. Host-defense peptides are present in almost every life form and are an integrated part of the eukaryotic immune system mounting a first-line defense against pathogens. These peptides can have both a direct killing activity and immunomodulatory properties. Sec, e.g., Hancock, R. E. “Cationic peptides: effectors in innate immunity and novel antimicrobials.” Lancet Infect. Dis., 2001, 1(3), 156-164. Several cationic host-defense peptides have shown anti-cancer activity due to their ability to bind to negatively charged membrane components on cells via electrostatic interactions. Sec, e.g., Zasloff, M. “Antimicrobial Peptides in Health and Disease.” New England Journal of Medicine, 2002, 347(15), 1199-1200; and Papo et al. “Host defense peptides as new weapons in cancer treatment.” Cell Mol. Life Sci. 2005, 62(7-8), 784-790.

Without wishing to be bound by any particular theory, Peptide A can enhance the cytolytic activity of naturally occurring host-defense peptides, subsequently inducing lysis and cell death at high local concentration. See, e.g., Forveille et al. “The oncolytic peptide LTX-315 triggers necrotic cell death.” Cell Cycle, 2015, 14(21), 3506-3512. It is believed that Peptide A induces immunogenic cell death, killing the cells in a manner that activates the adaptive immune system. It is also believed that a tumor-specific immune response is activated due to an effective release of potent immune stimulants (i.e., danger signals) and a broad repertoire of tumor antigens. Sec, e.g., Sveinbjørnsson et al. “LTX-315: a first-in-class oncolytic peptide that reprograms the tumor microenvironment.” Future Med. Chem., 2017, 9(12), 1339-1344; Jebsen et al. “Enhanced T-lymphocyte infiltration in a desmoid tumor of the thoracic wall in a young woman treated with intratumoral injections of the oncolytic peptide LTX-315: a case report,” J. Med. Case Rep., 2019, 13(1), 177; Liao et al. “LTX-315 sequentially promotes lymphocyte-independent and lymphocyte-dependent antitumor effects.” Cell Stress, 2019, 3(11), 348-360; Nestvold et al. “Oncolytic peptide LTX-315 induces an immune-mediated abscopal effect in a rat sarcoma model.” Oncoimmunology, 2017, 6(8), c1338236; Camilio et al. “Complete regression and systemic protective immune responses obtained in B16 melanomas after treatment with LTX-315.” Cancer Immunol. Immunother., 2014, 63(6), 601-613; and Spicer et al. “Safety, Antitumor Activity, and T-cell Responses in a Dose-Ranging Phase I Trial of the Oncolytic Peptide LTX-315 in Patients with Solid Tumors.” Clin. Cancer Res., 2021, 27(10), 2755-2763. As described herein, the present disclosure is based on the development of improved clinical protocols for administering Peptide A for the treatment of skin cancer (e.g., basal cell carcinoma).

### Pharmaceutical Compositions, Dosing, and Administration

Methods provided herein refer to administering a total volume of a pharmaceutical composition comprising Peptide A to a subject. As used herein, “total volume” refers to the total volume of the pharmaceutical composition to be administered to the subject over the course of a treatment session (i.e., over the course of method steps (a)-(c) for partitioned administration, over the course of method steps (a) and (b) for radial administration, or over the course of method steps (a)-(c) for partitioned and radial administration), rather than the volume of the pharmaceutical composition present in a syringe or vial. In some instances, the total volume of the pharmaceutical composition to be administered to the subject is less than the volume present in a syringe or vial. In certain embodiments, the “total volume” is the volume of the pharmaceutical composition to be administered to the subject in one day.

In some embodiments, the total volume of the pharmaceutical composition is from 10 μL to 2000 μL, inclusive. In some embodiments, the total volume of the pharmaceutical composition is from 10 μL to 1000 μL, inclusive. In some embodiments, the total volume of the pharmaceutical composition is from 100 μL to 1000 μL, inclusive. In some embodiments, the total volume of the pharmaceutical composition is from 200 μL to 800 μL, inclusive. In some embodiments, the total volume of the pharmaceutical composition is from 400 μL to 600 μL, inclusive. In some embodiments, the total volume of the pharmaceutical composition is approximately 100, 200, 300, 400, 500, 600, 700, 800, 900, or 1000 μL. In some embodiments, the total volume of the pharmaceutical composition is approximately 500 μL.

In some embodiments, the total volume of the pharmaceutical composition comprises an effective amount (e.g., a therapeutically effective amount) of Peptide A. In some embodiments, the total volume of the pharmaceutical composition comprises a single dose (e.g., daily dose) of Peptide A, and in such instances may be referred to as the “dosage volume.”

In some embodiments, a single dose of Peptide A is less than or equal to 8 mg. In some embodiments, the total volume of the pharmaceutical composition comprises less than or equal to 8 mg of Peptide A. In some embodiments, the total volume of the pharmaceutical composition comprises from 2 mg to 8 mg, inclusive, of Peptide A. In some embodiments, the total volume of the pharmaceutical composition comprises from 4 mg to 8 mg, inclusive, of Peptide A. In some embodiments, the total volume of the pharmaceutical composition comprises from 6 mg to 8 mg, inclusive, of Peptide A. In some embodiments, the total volume of the pharmaceutical composition comprises approximately 8 mg of Peptide A.

In some embodiments, the concentration of Peptide A in the pharmaceutical composition is from 10 mg/mL to 20 mg/mL, inclusive. In some embodiments, the concentration of Peptide A in the pharmaceutical composition is from 14 mg/mL to 18 mg/mL, inclusive. In some embodiments, the concentration of Peptide A in the pharmaceutical composition is approximately 16 mg/mL (i.e., approximately 8 mg of Peptide A in approximately 500 μL of solution).

Pharmaceutical compositions described herein comprise Peptide A, one or more liquid carriers, and optionally one or more pharmaceutically acceptable excipients. Pharmaceutically acceptable carriers/excipients used in pharmaceutical compositions include inert diluents, solvents, dispersing agents, surface active agents, emulsifiers, disintegrating agents, binding agents, preservatives, and buffering agents.

In some embodiments, the pharmaceutical composition is an aqueous composition. In some embodiments, the pharmaceutical composition comprises Peptide A and normal saline (0.9% aqueous sodium chloride (NaCl)). In some embodiments, the pharmaceutical composition consists essentially of Peptide A and normal saline. In some embodiments, the pharmaceutical composition consists of Peptide A and normal saline.

In some embodiments, a method provided herein is performed on a subject once daily. In some embodiments, a method provided herein is performed once daily for 2-3 consecutive days per week.

In some embodiments, a method provided herein is performed on a subject once daily for two consecutive days. In some embodiments, a method provided herein is performed on a subject once daily for two consecutive days, followed by one or more consecutive days (e.g., 1, 2, 3, 4, 5, 6, 7 consecutive days) of no treatment with Peptide A (referred to as a “treatment cycle”). For example, in some embodiments, a method provided herein is performed on a subject once daily for two consecutive days, followed by five consecutive days of no treatment with Peptide A (e.g., once daily for two consecutive days per week). In certain embodiments, a treatment cycle is be repeated one or more times (e.g., 1, 2, 3, 4, 5, 6, or 7 more times) until a desired outcome is achieved, e.g., until the skin cancer lesion has necrosed, reduced in size, or cleared.

In some embodiments, a method provided herein is performed on a subject once daily, wherein no more than 8 mg (e.g., 2 mg to 8 mg, inclusive) of Peptide A is administered to the subject per day. In some embodiments, a method provided herein is performed on a subject once daily for two consecutive days, wherein no more than 8 mg (e.g., 2 mg to 8 mg, inclusive) of Peptide A is administered to the subject per day. In some embodiments, a method provided herein is performed on a subject once daily for two consecutive days, wherein no more than 8 mg (e.g., 2 mg to 8 mg, inclusive) of Peptide A is administered to the subject per day, followed by one or more consecutive days (e.g., 1, 2, 3, 4, 5, 6, 7 consecutive days) of no treatment with Peptide A (referred to as a “treatment cycle”). For example, in some embodiments, a method provided herein is performed on a subject once daily for two consecutive days, wherein no more than 8 mg (e.g., 2 mg to 8 mg, inclusive) of Peptide A is administered to the subject per day, followed by five consecutive days of no treatment with Peptide A (e.g., once daily for two consecutive days per week). In certain embodiments, a treatment cycle is be repeated one or more times (e.g., 1, 2, 3, 4, 5, 6, or 7 more times) until a desired outcome is achieved e.g., until the skin cancer lesion has necrosed, reduced in size, or cleared.

In some embodiments, a method provided herein is performed on a subject once daily for three consecutive days. In some embodiments, a method provided herein is performed on a subject once daily for three consecutive days, followed by one or more consecutive days (e.g., 1, 2, 3, 4, 5, 6, or 7 consecutive days) of no treatment with Peptide A (referred to as a “treatment cycle”). For example, in some embodiments, a method provided herein is performed on a subject once daily for three consecutive days, followed by four consecutive days of no treatment with Peptide A (e.g., once daily for three consecutive days per week). In certain embodiments, a treatment cycle is be repeated one or more times (e.g., 1, 2, 3, 4, 5, 6, or 7 more times) until a desired outcome is achieved e.g., until the skin cancer lesion has necrosed, reduced in size, or cleared.

In some embodiments, a method provided herein is performed on a subject once daily for three consecutive days, wherein no more than 8 mg (e.g., 2 mg to 8 mg, inclusive) of Peptide A is administered to the subject per day. In some embodiments, a method provided herein is performed on a subject once daily for three consecutive days, wherein no more than 8 mg (e.g., 2 mg to 8 mg, inclusive) of Peptide A is administered to the subject per day, followed by one or more consecutive days (e.g., 1, 2, 3, 4, 5, 6, 7 consecutive days) of no treatment with Peptide A (referred to as a “treatment cycle”). For example, in some embodiments, a method provided herein is performed on a subject once daily for three consecutive days, wherein no more than 8 mg (e.g., 2 mg to 8 mg, inclusive) of Peptide A is administered to the subject per day, followed by four consecutive days of no treatment with Peptide A (e.g., once daily for three consecutive days per week). In certain embodiments, a treatment cycle is be repeated one or more times (e.g., 1, 2, 3, 4, 5, 6, or 7 more times) until a desired outcome is achieved e.g., until the skin cancer lesion has necrosed, reduced in size, or cleared.

In some embodiments, the pharmaceutical composition is injected using a syringe with a 30-gauge needle (e.g., a 30-gauge, 0.5 inch needle). In certain embodiments, the syringe is a beveled needle. In cases where the skin cancer lesion (e.g., BCC skin lesion) is a flat lesion, the pharmaceutical composition can be injected at a 10- to 20-degree angle, inclusive. In cases where the skin cancer lesion (e.g., BCC skin lesion) is a raised lesion, the pharmaceutical composition can be injected at an angle greater than 20 degrees. A physician using sound medical judgement can choose the appropriate syringe, needle, and injection angle when carrying out a method provided herein.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In preferred embodiments, the subject is a human. In preferred embodiments, the subject is an adult human (18 years of age or older).

The term “administer,” “administering,” or “administration” refers, for the purposes of this disclosure, to injecting a pharmaceutical composition comprising Peptide A into the skin of a subject. In some embodiments, the pharmaceutical composition is administered via intradermal injection into the skin of the subject, e.g., into a skin cancer lesion.

“Intradermal,” “intradermally,” and the like refer to a shallow or superficial injection into the dermis of the skin, which is located between the epidermis and the hypodermis of the skin.

The terms “treatment,” “treat,” and “treating” refer to reversing, alleviating, delaying the onset of, or inhibiting the progress of a disease described herein (e.g., a skin cancer, such as basal cell carcinoma).

An “effective amount” of Peptide A or a pharmaceutical composition thereof described herein refers to an amount sufficient to elicit the desired biological response (e.g., treatment of a skin cancer lesion, e.g., a BCC skin lesion). A “therapeutically effective amount” of Peptide A or a pharmaceutical composition thereof is an amount sufficient to provide a therapeutic benefit in the treatment of a condition or to delay or minimize one or more symptoms associated with the condition (i.e., treatment of a skin cancer lesion, e.g., a BCC skin lesion). A therapeutically effective amount means an amount of Peptide A or pharmaceutical composition thereof, alone or in combination with other therapies, which provides a therapeutic benefit in the treatment of the condition (i.e., treatment of a skin cancer lesion, e.g., a BCC skin lesion).

As used herein, “treatment-related adverse event” (TRAE) refers to any adverse event (i.e., negative side effect) that in a physician's opinion is caused by the study medication (for the purposes of this disclosure, Peptide A). Treatment-related adverse events (TRAEs) can include one or more of pain, burning, and/or stinging at or around the injection site.

### Skin Cancer and Lesions

Provided herein are methods and compositions for treating skin cancer. Non-limiting examples of skin cancers include basal cell carcinoma (BCC), squamous cell carcinoma (SCC), melanoma, and Merkel cell carcinoma (MCC). In some embodiments, the skin cancer is non-metastatic, for example, non-metastatic basal cell carcinoma (BCC), non-metastatic squamous cell carcinoma (SCC), non-metastatic melanoma, and non-metastatic Merkel cell carcinoma (MCC). In some embodiments, the skin cancer is non-metastatic BCC. “Non-metastatic” in the context of cancer refers to a cancer or tumor which has not spread from its original site (i.e., the skin) to other parts of the body.

In some embodiments, the skin cancer is non-advanced. “Non-advanced” skin cancer is skin cancer characterized by lesion(s) less than or equal to 2 centimeters (cm) in the longest diameter. In some embodiments, the skin cancer is non-advanced basal cell carcinoma (BCC), non-advanced squamous cell carcinoma (SCC), non-advanced melanoma, or non-advanced Merkel cell carcinoma (MCC). In some embodiments, the skin cancer is non-advanced BCC.

In some embodiments, a skin cancer lesion is from 0.5 cm to 2.0 cm, inclusive, in the longest diameter. In some embodiments, a skin cancer lesion is from 1 cm to 2.0 cm, inclusive, in the longest diameter. In some embodiments, a skin cancer lesion is from 1.5 cm to 2.0 cm, inclusive, in the longest diameter. For example, in some embodiments, a BCC skin lesion is from 0.5 cm to 2.0 cm, inclusive, in the longest diameter. In some embodiments, a BCC skin lesion is from 1 cm to 2.0 cm, inclusive, in the longest diameter. In some embodiments, a BCC skin lesion is from 1.5 cm to 2.0 cm, inclusive, in the longest diameter. In some embodiments, a skin cancer lesion (e.g., BCC skin lesion) is 0.5 cm or greater in its longest diameter.

The terms “tumor” and “lesion” when referring to a skin cancer (e.g., BCC) are used interchangeably herein.

In certain embodiments, treating skin cancer (e.g., BCC) can result in a reduction in size or volume of a skin cancer lesion (e.g., BCC skin lesion). For example, after treatment, lesion size is reduced by 5% or greater (e.g., 10%, 20%, 30%, 40%, 50%, 60%, 70%, 80%, 90% or greater) relative to its size prior to treatment. In some embodiments, the skin cancer lesion (e.g., BCC skin lesion) is cleared. The size of a skin cancer lesion (e.g., BCC skin lesion) may be measured as a diameter of the lesion or by any reproducible means of measurement. In certain embodiments, treating skin cancer (e.g., BCC) can result in a reduction in the rate of growth or metastasis of the cancer. For example, after treatment, the rate of growth is reduced by 5% or greater (e.g., 10%, 20%, 30%, 40%, 50%, 60%, 70%, 80%, 90% or greater) relative to the rate of growth prior to treatment.

### ADDITIONAL EMBODIMENTS

Additional embodiments are provided according to the following numbered Embodiments:
Embodiment 1. A method of treating basal cell carcinoma (BCC) in a subject in need thereof, the method comprising the following sequential steps:
(a) injecting intradermally 20%-40%, inclusive, of the total volume of a pharmaceutical composition comprising Peptide A into a BCC skin lesion of the subject;
(b) waiting between 5 and 60 minutes, inclusive; and
(c) injecting intradermally the remaining 60%-80%, inclusive, of the total volume of the pharmaceutical composition into the BCC skin lesion,
wherein Peptide A is KKWWKKWDipK-NH₂ (SEQ ID NO: 1), or a pharmaceutically acceptable salt thereof.
Embodiment 2. The method of Embodiment 1, wherein about 30% of the total volume of the pharmaceutical composition is injected into the BCC skin lesion in step (a); and the remaining about 70% is injected into the BCC skin lesion in step (c).
Embodiment 3. The method of Embodiment 1 or 2, wherein step (b) comprises waiting between 5 and 30 minutes, inclusive.
Embodiment 4. The method of any one of Embodiments 1-3, wherein step (b) comprises waiting between 15 and 30 minutes, inclusive.
Embodiment 5. A method of treating basal cell carcinoma (BCC) in a subject in need thereof, the method comprising the following sequential steps:
(a) injecting intradermally a portion of the total volume of a pharmaceutical composition comprising Peptide A into the center of a BCC skin lesion of the subject; and
(b) injecting intradermally the remaining portion of the total volume of the pharmaceutical composition into 2 or more sites at the periphery of the BCC skin lesion,
wherein Peptide A is KKWWKKWDipK-NH₂ (SEQ ID NO: 1), or a pharmaceutically acceptable salt thereof.
Embodiment 6. The method of Embodiment 5, wherein step (b) comprises injecting the remaining portion of the total volume of the pharmaceutical composition into 3 or more sites at the periphery of the BCC skin lesion.
Embodiment 7. The method of Embodiment 5, wherein step (b) comprises injecting the remaining portion of the total volume of the pharmaceutical composition into 4 or more sites at the periphery of the BCC skin lesion.
Embodiment 8. The method of Embodiment 5, wherein step (b) comprises injecting the remaining portion of the total volume of the pharmaceutical composition into 2-6 sites at the periphery of the BCC skin lesion.
Embodiment 9. The method of Embodiment 5, wherein step (b) comprises injecting the remaining portion of the total volume of the pharmaceutical composition into 3-5 sites at the periphery of the BCC skin lesion.
Embodiment 10. The method of Embodiment 5, wherein step (b) comprises injecting the remaining portion of the total volume of the pharmaceutical composition into 4 sites at the periphery of the BCC skin lesion.
Embodiment 11. The method of any one of Embodiments 5-10, wherein the sites at the periphery of the BCC skin lesion are approximately equidistant from one another.
Embodiment 12. A method of treating basal cell carcinoma (BCC) in a subject in need thereof the method comprising the following sequential steps:
(a) injecting intradermally 20%-40%, inclusive, of the total volume of a pharmaceutical composition comprising Peptide A into the center of a BCC skin lesion of the subject;
(b) waiting between 5 and 60 minutes, inclusive; and
(c) injecting intradermally the remaining 60%-80%, inclusive, of the total volume of the pharmaceutical composition to 2 or more sites at the periphery of the BCC skin lesion,
wherein Peptide A is KKWWKKWDipK-NH₂ (SEQ ID NO: 1), or a pharmaceutically acceptable salt thereof.
Embodiment 13. The method of Embodiment 12, wherein about 30% of the total volume of the pharmaceutical composition is injected into the BCC skin lesion in step (a); and the remaining about 70% is injected into the BCC skin lesion in step (c).
Embodiment 14. The method of Embodiment 12 or 13, wherein step (b) comprises waiting between 5 and 30 minutes, inclusive.
Embodiment 15. The method of any one of Embodiments 12-14, wherein step (b) comprises waiting between 15 and 30 minutes, inclusive.
Embodiment 16. The method of any one of Embodiments 12-15, wherein step (c) comprises injecting the remaining portion of the total volume of the pharmaceutical composition into 3 or more sites at the periphery of the BCC skin lesion.
Embodiment 17. The method of any one of Embodiments 12-15, wherein step (c) comprises injecting the remaining portion of the total volume of the pharmaceutical composition into 4 or more sites at the periphery of the BCC skin lesion.
Embodiment 18. The method of any one of Embodiments 12-15, wherein step (c) comprises injecting the remaining portion of the total volume of the pharmaceutical composition into 2-6 sites at the periphery of the BCC skin lesion.
Embodiment 19. The method of any one of Embodiments 12-15, wherein step (c) comprises injecting the remaining portion of the total volume of the pharmaceutical composition into 3-5 sites at the periphery of the BCC skin lesion.
Embodiment 20. The method of any one of Embodiments 12-15, wherein step (c) comprises injecting the remaining portion of the total volume of the pharmaceutical composition into 4 sites at the periphery of the BCC skin lesion.
Embodiment 21. The method of any one of Embodiments 12-20, wherein the sites at the periphery of the BCC skin lesion are approximately equidistant from one another.
Embodiment 22. The method of any one of the preceding Embodiments, wherein the total volume of the pharmaceutical composition is from 100 μL to 1000 μL, inclusive.
Embodiment 23. The method of any one of the preceding Embodiments, wherein the total volume of the pharmaceutical composition is from 400 μL to 600 μL, inclusive.
Embodiment 24. The method of any one of the preceding Embodiments, wherein the total volume of the pharmaceutical composition is approximately 500 μL.
Embodiment 25. The method of any one of the preceding Embodiments, wherein the total volume of the pharmaceutical composition comprises from 2 mg to 8 mg, inclusive, of Peptide A.
Embodiment 26. The method of any one of the preceding Embodiments, wherein the total volume of the pharmaceutical composition comprises approximately 8 mg of Peptide A.
Embodiment 27. The method of any one of the preceding Embodiments, wherein the concentration of Peptide A in the pharmaceutical composition is from 10 mg/mL to 20 mg/mL, inclusive.
Embodiment 28. The method of any one of the preceding Embodiments, wherein the concentration of Peptide A in the pharmaceutical composition is from 14 mg/mL to 18 mg/mL, inclusive.
Embodiment 29. The method of any one of the preceding Embodiments, wherein the concentration of Peptide A in the pharmaceutical composition is approximately 16 mg/mL.
Embodiment 30. The method of any one of the preceding Embodiments, wherein the pharmaceutical composition comprises Peptide A and normal saline.
Embodiment 31. The method of any one of the preceding Embodiments, wherein the method is performed once daily.
Embodiment 32. The method of any one of the preceding Embodiments, wherein the method is performed once daily for three consecutive days.
Embodiment 33. The method of any one of the preceding Embodiments, wherein the method is performed once daily for two consecutive days.
Embodiment 34. The method of any one of the preceding Embodiments, wherein the method is performed once daily for two consecutive days, followed by five consecutive days of no treatment with Peptide A.
Embodiment 35. The method of any one of the preceding Embodiments, wherein the method is performed once daily for three consecutive days, followed by four consecutive days of no treatment with Peptide A.
Embodiment 36. The method of any one of the preceding Embodiments, wherein no more than 8 mg of Peptide A is administered to the subject per day.
Embodiment 37. The method of any one of the preceding Embodiments, wherein the pharmaceutical composition is injected using a syringe with a 30-gauge needle.
Embodiment 38. The method of any one of the preceding Embodiments, wherein the pharmaceutical composition is injected using a syringe with a 30-gauge, 0.5 inch needle.
Embodiment 39. The method of any one of the preceding Embodiments, wherein the pharmaceutical composition is injected using a syringe with a beveled needle.
Embodiment 40. The method of any one of the preceding Embodiments, wherein the BCC skin lesion is a flat lesion, and the pharmaceutical composition is injected at a 10- to 20-degree angle, inclusive.
Embodiment 41. The method of any one of the preceding Embodiments, wherein the BCC skin lesion is a raised lesion, and the pharmaceutical composition is injected at a greater than 20-degree angle.
Embodiment 42. The method of any one of the preceding Embodiments, wherein the BCC is non-advanced basal cell carcinoma.
Embodiment 43. The method of any one of the preceding Embodiments, wherein the BCC skin lesion is from 0.5 cm to 2.0 cm, inclusive, in its longest diameter.
Embodiment 44. The method of any one of the preceding Embodiments, wherein Peptide A is the acetate salt of KKWWKKWDipK-NH₂ (SEQ ID NO: 1).
Embodiment 45. The method of any one of Embodiments 1-43, wherein the subject is a human.
Embodiment 46. A pharmaceutical composition comprising Peptide A for use in treating basal cell carcinoma (BCC) according to a method of any one of the preceding Embodiments.
Embodiment 47. Use a pharmaceutical composition comprising Peptide A as a medicament treating basal cell carcinoma (BCC) according to a method of any one of the preceding Embodiments.

### EXAMPLES

### Example 1. Clinical Protocol for Treating Basal Cell Carcinoma Lesion(s) with Formulations Comprising Peptide A

i. Study Inclusion Criteria

This study involved human adults ≥18 years of age with clinically suspected BCC with at least 1 (and up to 5) eligible lesion(s) suitable for biopsy and excision. The size of the lesion(s) were ≥0.5 cm and ≤2 cm in longest diameter prior to punch biopsy or ≥1 cm and ≤2 cm in longest diameter prior to shave biopsy. Histological diagnosis of nodular, micronodular, or superficial BCC, as confirmed by punch or shave biopsy, was performed within 90 days of treatment.

ii. Formulation, Storage, and Handling

Peptide A was provided as the acetate salt, 20 mg of lyophilized powder per vial for injection. The powder was reconstituted in saline to a concentration ranging from 4-20 mg/mL based upon the target dose and volume for each subject. Peptide A was supplied in colorless glass vials (Ph. Eur. hydrolytic class I) fitted with rubber stopper (Ph. Eur. type I).

Peptide A vials (20 mg/vial) were stored at 2-8° C. (36-46° F.) or −20±5° C. (−13 to 5° F.) protected from light at the study site, separated from other drugs. The reconstituted Peptide A solution can be stored for up to 12 hours in a refrigerator at 2-8° C. (36-46° F.). Syringes with Peptide A drawn from the vial can be stored for up to 4 hours at 20-25° C. (68-77° F.) protected from light. Any unused portion was discarded.

iii. Administration Protocol

Peptide A (acetate salt) was administered via intradermal injection into a single target lesion (between ≥0.5 cm and ≤2 cm in longest diameter) using the 1-mL syringe and 30-gauge 0.5-inch needle. The subjects were positioned in a way so that the target lesion lies flat, to avoid any dependent areas that may be susceptible to leakage of the pharmaceutical composition. Subsequent injections for the same lesion were given in remaining nonnecrotic areas of the lesion, as assessed prior to treatment.

Each 500-μL dose (8 mg Peptide A acetate salt in normal saline) was divided into 2 injections given at least 15 minutes and no more than 30 minutes apart, with 30% (150 μL) administered in the first injection and the remaining 70% (350 μL) with the second injection. The targeted total volume of delivery was 500 μL (8 mg Peptide A acetate salt) daily.

For all injections, the pharmaceutical composition was injected intradermally. It is critical to assure adequate and even distribution of the pharmaceutical composition across the entire base of the lesion. To accomplish this, the first 150 μL was injected as centrally as possible such that the pharmaceutical composition can distribute radially outward (i.e., at position C in FIG. 1). The second (350 μL) portion was injected in a clockface fashion (i.e., at positions 12, 3, 6, and 9 o'clock in FIG. 1) as needed to ensure uniform distribution, into the edges of the lesion to infiltrate the entire tumor with the pharmaceutical composition. The needle was inserted at a 10- to 20-degree angle for a flat lesion, or at an increased angle for a raised lesion. Subcutaneous injection was avoided. The maximum total daily dose of Peptide A in any treatment visit in any subject or cohort did not exceed 8 mg.

### iv. 2- and 3-Day Dosing Studies

For the clinical protocol described above, subjects are split into cohorts based on 2-day and 3-day dosing regimens (“two times weekly” and “three times weekly”, respectively). The highest total daily dose of Peptide A (8.0 mg) is divided into a split dose; the first dose is not to exceed 2.4 mg (30% of 8 mg dose), and the remaining dose is not to exceed 5.6 mg (70% of 8 mg dose). The first and second dose are administered 15 minutes apart (not to exceed 30 minutes).
Two times weekly dosing: Peptide A once-daily dosing of 8 mg, administered on 2 consecutive days in one week. The dosing regimen is a split dose of Peptide A for all treatments. The 500 μL (8 mg) dose is divided into 2 injections given at least 15 minutes and no more than 30 minutes apart, with 30% (150 μL) administered in the first injection and the remaining 70% (350 μL) with the second injection. Treatment for a second target lesion may begin on day 1 of the following week. Each individual target lesion is treated for the assigned 2 days only (regardless of necrosis status).
Three times weekly dosing: Peptide A once-daily dosing of 8 mg, administered on 3 consecutive days in one week. The planned dosing regimen is a split dose of Peptide A for all treatments. The 500 μL (8 mg) dose is divided into 2 injections given at least 15 minutes and no more than 30 minutes apart, with 30% (150 μL) administered in the first injection and the remaining 70% (350 μL) with the second injection. Treatment for a second target lesion may begin on day 1 of the following week. Each individual target lesion is treated for the assigned 3 days only (regardless of necrosis status).
   v. Pharmacokinetic (PK) Analysis

Blood samples were collected on the first day of dose administration immediately prior to the Peptide A dose and at 2- and 5-minutes post first injection, and 2-, 5-, 10-, 20-, 30-, 60-, and 120-minutes post last injection. Additional samples were collected at 24 (±1) hours post-dose prior to administration of the next Peptide A dose.

Concentrations of Peptide A and metabolites are determined in plasma using a validated bioanalytical assay(s). Individual subject Peptide A concentration-time data can be displayed using scheduled sampling times. Descriptive statistics (e.g., n, mean, SD, % CV, median and range) can be calculated for each sampling time. Plasma concentrations of the study drug over time can be plotted in semilogarithmic and linear formats as mean±SD. Plasma concentration-time data for each subject can be analyzed using standard noncompartmental methods. Plasma PK parameters of Peptide A and metabolites will include Cₘₐₓ, Tₘₐₓ, AUC₀₋ₗₐₛₜ, and AUC_{inf}.

Dose proportionality information can be obtained by comparing plasma levels of Peptide A across all dose levels. The primary method for evaluation of dose proportionality can be based on AUC₀₋ₗₐₛₜ, AUC_{inf}, AUCₜₐᵤ, and Cₘₐₓ. The population mean slope can be estimated with a 90% CI. An alternate evaluation of dose proportionality can be conducted using ANOVA on dose-normalized AUC₀₋ₗₐₛₜ, AUC_{inf}, and Cₘₐₓ. The analysis can be conducted on log-transformed data. The model can include dose as a fixed effect and subject as random effect. Calculations of the 90% CI for the ratio of dose comparisons will be constructed.

vi. Clinical Data

Subjects represented in Table 1 below did not receive partitioned administration of Peptide A. Cohort 1 was assigned a regimen of 4 mg of Peptide A on day 1 (W1D1), followed by 8 mg of Peptide A on subsequent days (with no partitioned administration on any day). Cohort 2 was assigned a regimen of 8 mg of Peptide A for each treatment day (with no partitioned administration on any day). Several subjects in Cohort 1 and 2 experienced treatment-related adverse events (TRAEs) of burning and pain at the injection site (see Table 1). Two subjects withdrew from treatment due to TRAEs.

TABLE 1

| | | Dose of Peptide A | | | | | | |
|---|---|---|---|---|---|---|---|---|
| Subject | Cohort | W1D1 | W1D2 | W1D3 | W2D1 | W2D2 | W2D3 | TRAEs |
| 1 | Cohort 1 | 4 mg | 8 mg | 8 mg | 8 mg | 8 mg | 8 mg | |
| 2 | Cohort 1 | 4 mg | 8 mg | 8 mg | 8 mg | 8 mg | 8 mg | |
| 3 | Cohort 1 | 4 mg | Discontinued | | N/A | | | Subject 3 experienced |
| | | | | | | | | severe pain during and |
| | | | | | | | | after intratumoral |
| | | | | | | | | injection of 4 mg of |
| | | | | | | | | Peptide A into the basal |
| | | | | | | | | cell carcinoma lesion on |
| | | | | | | | | the lower back. Subject |
| | | | | | | | | was withdrawn from |
| | | | | | | | | treatment. |
| 4 | Cohort 1 | 4 mg | 8 mg | 8 mg | N/A | | | |
| 5 | Cohort 1 | 4 mg | 8 mg | Complete | 8 mg | 8 mg | 8 mg | |
| | | | | necrosis, no | | | | |
| | | | | injection | | | | |
| 6 | Cohort 1 | 4 mg | 8 mg | 8 mg | 8 mg | 8 mg | 8 mg | |

| 7 | Cohort 2 | 8 mg | 8 mg | 8 mg | N/A | |
|---|---|---|---|---|---|---|
| 8 | Cohort 2 | 8 mg | Discontinued | | N/A | Subject 8 withdrew |
| | | | | | | from treatment on |
| | | | | | | W1D2 after |
| | | | | | | experiencing moderate |
| | | | | | | pain. The pain lasted for |
| | | | | | | about 3-4 hours post- |
| | | | | | | treatment. |
| 9 | Cohort 2 | 8 mg | 8 mg | 8 mg | N/A | Subject 9 reported |

injection-site burning
after the W1D2
treatment. The subject
continued with
treatment despite
TRAE.

Based on these results, the clinical protocol was changed to adopt a partitioned administration method. For Cohorts 4 and 5 (Table 2 below), the dosing regimen is a split dose of Peptide A for all treatments. The total 500 μL (8 mg) dosage volume is divided into 2 injections given at least 15 minutes and no more than 30 minutes apart, with 30% (150 μL) administered in the first injection and the remaining 70% (350 μL) with the second injection.

A radial administration method was also used for Cohorts 4 and 5. The formulation with Peptide A is injected intradermally for all injections and subcutaneous injection should be avoided. It is important to ensure adequate and even distribution of Peptide A across the entire base of the lesion. To accomplish adequate and even distribution of Peptide A across the entire lesion: The first 150 μL is injected as centrally as possible such that Peptide A can distribute radially outward; and the second (350 μL) portion is injected in a clockface fashion (i.e., at 12, 3, 6, 9 o'clock as shown in FIG. 1) as needed to ensure uniform distribution into the periphery of the lesion. The needle is inserted at a 10- to 20-degree angle for a flat lesion, or at an increased angle (i.e., greater than 20-degree) for a raised lesion.

Cohorts 4 and 5, subject to partitioned and radial administration of Peptide A, saw a marked decrease in TRAEs. As shown in Table 2, no subjects from Cohorts 4 and 5 (including from the expansion cohorts) have withdrawn from study treatments due to TRAEs of burning or pain at the injection site.

TABLE 2

| | | Subjects | |
|---|---|---|---|
| Study | Cohort | Enrolled | Results |
| 2a | Cohort 4 | 10 | No subjects have withdrawn from study |
| 2b | Cohort 5 | 10 | treatment due to TRAEs of burning or |
| 2c | Cohort 4 - Expansion | 26 | pain at the injection site |
| 2d | Cohort 5 - Expansion | 27 | |

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A method of treating basal cell carcinoma (BCC) in a subject in need thereof, the method comprising the following sequential steps:
(a) injecting intradermally 20%-40%, inclusive, of the total volume of a pharmaceutical composition comprising Peptide A into a BCC skin lesion of the subject;
(b) waiting between 5 and 60 minutes, inclusive; and
(c) injecting intradermally the remaining 60%-80%, inclusive, of the total volume of the pharmaceutical composition into the BCC skin lesion,
wherein Peptide A is KKWWKKWDipK-NH₂ (SEQ ID NO: 1), or a pharmaceutically acceptable salt thereof.

2-4. (canceled)

5. A method of treating basal cell carcinoma (BCC) in a subject in need thereof, the method comprising the following sequential steps:
(a) injecting intradermally a portion of the total volume of a pharmaceutical composition comprising Peptide A into the center of a BCC skin lesion of the subject; and
(b) injecting intradermally the remaining portion of the total volume of the pharmaceutical composition into 2 or more sites at the periphery of the BCC skin lesion,
wherein Peptide A is KKWWKKWDipK-NH₂ (SEQ ID NO: 1), or a pharmaceutically acceptable salt thereof.

6-11. (canceled)

12. A method of treating basal cell carcinoma (BCC) in a subject in need thereof the method comprising the following sequential steps:
(a) injecting intradermally 20%-40%, inclusive, of the total volume of a pharmaceutical composition comprising Peptide A into the center of a BCC skin lesion of the subject;
(b) waiting between 5 and 60 minutes, inclusive; and
(c) injecting intradermally the remaining 60%-80%, inclusive, of the total volume of the pharmaceutical composition to 2 or more sites at the periphery of the BCC skin lesion,
wherein Peptide A is KKWWKKWDipK-NH₂ (SEQ ID NO: 1), or a pharmaceutically acceptable salt thereof.

13. The method of claim 12, wherein about 30% of the total volume of the pharmaceutical composition is injected into the BCC skin lesion in step (a); and the remaining about 70% is injected into the BCC skin lesion in step (c).

14. The method of claim 12, wherein step (b) comprises waiting between 5 and 30 minutes, inclusive.

15. The method of claim 12, wherein step (b) comprises waiting between 15 and 30 minutes, inclusive.

16. The method of claim 12, wherein step (c) comprises injecting the remaining portion of the total volume of the pharmaceutical composition into 3 or more sites at the periphery of the BCC skin lesion.

17. (canceled)

18. The method of claim 12, wherein step (c) comprises injecting the remaining portion of the total volume of the pharmaceutical composition into 2-6 sites at the periphery of the BCC skin lesion.

19-20. (canceled)

21. The method of claim 12, wherein the sites at the periphery of the BCC skin lesion are approximately equidistant from one another.

22. The method of claim 12, wherein the total volume of the pharmaceutical composition is from 100 μL to 1000 μL, inclusive.

23. (canceled)

24. The method of claim 12, wherein the total volume of the pharmaceutical composition is approximately 500 μL.

25. The method of claim 12, wherein the total volume of the pharmaceutical composition comprises from 2 mg to 8 mg, inclusive, of Peptide A.

26. The method of claim 12, wherein the total volume of the pharmaceutical composition comprises approximately 8 mg of Peptide A.

27. The method of claim 12, wherein the concentration of Peptide A in the pharmaceutical composition is from 10 mg/mL to 20 mg/mL, inclusive.

28-29. (canceled)

30. The method of claim 12, wherein the pharmaceutical composition comprises Peptide A and normal saline.

31. The method of claim 12, wherein the method is performed once daily.

32. The method of claim 31, wherein the method is performed once daily for three consecutive days.

33. The method of claim 31, wherein the method is performed once daily for two consecutive days.

34. The method of claim 31, wherein the method is performed once daily for two consecutive days, followed by five consecutive days of no treatment with Peptide A.

35. The method of claim 31, wherein the method is performed once daily for three consecutive days, followed by four consecutive days of no treatment with Peptide A.

36. The method of claim 12, wherein no more than 8 mg of Peptide A is administered to the subject per day.

37-41. (canceled)

42. The method of claim 12, wherein the BCC is non-advanced basal cell carcinoma.

43. (canceled)

44. The method of claim 12, wherein Peptide A is the acetate salt of KKWWKKWDipK-NH₂ (SEQ ID NO: 1).

45. The method of claim 12, wherein the subject is a human.

46-47. (canceled)
